(19) United States
(12) Patent Application Publication
(10) Pub. No.: US 2006/0228410 A1
(43) Pub. Date: Oct. 12, 2006

(54) Flavored taste-masked pharmaceutical formulation made using a one-step coating process

(76) Inventors: Hubert Dumont, Quebec (CA); Roch Thibert, Quebec (CA)

Correspondence Address: MERCK AND CO., INC, P O BOX 2000, RAHWAY, NJ, 07065-0907, US

(21) Appl. No.: 10/565,609

(22) PCT Filed: Aug. 10, 2004
(86) PCT No.: PCT/CA04/01483

§ 371 Date: Jan. 23, 2006

(30) Foreign Application Priority Data
Aug. 11, 2003 (US) 60494370

(51) Int. Cl. A61K 9/20; A61K 31/415

(52) U.S. Cl. 424/464; 514/406

(57) ABSTRACT

The present invention encompasses a flavored and taste-masked pharmaceutical composition comprising a plurality of pharmaceutically acceptable cores, such as microspheres, said pharmaceutically acceptable cores comprising etoricoxib, wherein the pharmaceutically acceptable cores are coated with a flavored taste-masking coating solution in a convenient one-step process.

### BACKGROUND OF THE INVENTION

The present invention provides for a novel flavored taste-masked pharmaceutical formulation that can be made by a convenient one-step process. For pediatric and geriatric patients who cannot swallow a tablet, alternate formulations, such as a liquid suspension or oral granule formulation, may be utilized to administer a drug. However, a significant drawback may exist if the active ingredient possesses an unpleasant taste. Taste-masked formulations to address this problem are well known in the art, but often do not have a pleasant taste of their own. Generally, taste improvement is provided by means of a granulation process that agglomerates a taste-masked active pharmaceutical ingredient (API) or API-containing particles with bulking material, flavoring and sweetening agents, with the help of a binder. The disadvantages of this method are: 1) additional process steps; and 2) use of bulking agent in the granulation increasing the risk of dose uniformity problems especially on process scale-up.

The present invention addresses these drawbacks through the use of a one-step process for flavoring and taste-masking that has the following advantages: 1) extension of the taste-masking coating process (reduces the number of process steps); 2) quantity of bulking agent are reduced; and 3) the excipients are sprayed on the API or API containing core.

### SUMMARY OF THE INVENTION

The present invention encompasses a flavored and taste-masked pharmaceutical composition comprising a plurality of pharmaceutically acceptable cores, such as microspheres, said pharmaceutically acceptable cores comprising an active pharmaceutical ingredient having etoricoxib, wherein the pharmaceutically acceptable cores are coated with a flavored taste-masking coating solution in a convenient one-step process.

### DETAILED DESCRIPTION OF THE INVENTION

The invention encompasses a flavored and taste-masked pharmaceutical composition comprising a plurality of pharmaceutically acceptable cores, said pharmaceutically acceptable cores comprising etoricoxib, wherein the pharmaceutically acceptable cores are coated with a flavored taste-masking coating solution in a one-step coating process, said flavored taste-masking coating solution comprising the following ingredients:
(a) at least one taste-masking agent and
(b) at least one sweetening agent or at least one flavoring agent or at least one of both,

An embodiment of the invention encompasses the pharmaceutical composition wherein the flavored taste-masking coating solution further comprises:
(a) at least one bulking agent, and
(b) at least one glidant.

Another embodiment of the invention encompasses the pharmaceutical composition wherein the pharmaceutically acceptable cores are microspheres.

Another embodiment of the invention encompasses the pharmaceutical composition wherein the flavored taste-masking coating solution comprises the following ingredients:
(a) hydroxypropylmethyl cellulose,
(b) polymethacrylate,
(c) mannitol,
(d) aspartame,
(e) artificial cherry flavor,
(f) monoglyceride, and
(g) water.

Another embodiment of the invention encompasses a flavored and taste-masked pharmaceutical composition comprising a plurality of microspheres, said microspheres comprising etoricoxib, wherein the microspheres are coated with a flavored taste-masking coating solution in a one-step coating process, the flavored taste-masking coating solution comprising the following ingredients:
(a) hydroxypropylmethyl cellulose,
(b) polymethacrylate,
(c) mannitol,
(d) aspartame,
(e) artificial cherry flavor,
(f) monoglyceride, and
(g) water.

Within this embodiment is encompassed the pharmaceutical composition wherein the ingredients in the flavored taste-masking solution are present in the following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |

Another embodiment of the invention encompasses the pharmaceutical composition prepared by a process comprising:
(1) preparing the flavored taste-masking coating solution by combining the following ingredients:
   (a) at least one taste-masking agent and
   (b) at least one sweetening agent or at least one flavoring agent or at least one of both, and
(2) coating the pharmaceutically acceptable cores with the flavored taste-masking coating solution in a one-step coating process. Within this embodiment is encompassed the pharmaceutical composition wherein step 1) for preparing the flavored taste-masking coating solution further comprises adding the following ingredients:
   (a) at least one bulking agent, and
   (b) at least one glidant.

Within this embodiment is encompassed the above pharmaceutical composition wherein the pharmaceutically acceptable cores are microspheres. Also within this embodiment is encompassed the above pharmaceutical composition wherein the flavored taste-masking coating solution is prepared by combining the following ingredients:
(a) hydroxypropylmethyl cellulose,
(b) polymethacrylate,
(c) mannitol,
(d) aspartame,
(e) artificial cherry flavor,
(f) monoglyceride, and
(g) water.
   Also within this embodiment is encompassed the above pharmaceutical composition wherein the flavored taste-masking coating solution is prepared as follows:
(a) dissolving hydroxypropylmethyl cellulose in deionized water;
(b) adding polymethacrylate and homogenizing; and

(c) adding mannitol, aspartame, artificial cherry flavor and monoglyceride and homogenizing. Also within this embodiment is encompassed the above pharmaceutical composition wherein the ingredients in the flavored taste-masking solution are combined to produce a solution having following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |

Also within this embodiment is encompassed the above pharmaceutical composition wherein the pharmaceutical cores are coated using a fluid bed system. Within this embodiment, the pharmaceutical cores are microspheres.

Another embodiment of the invention encompasses a flavored and taste-masked pharmaceutical composition comprising a plurality of microspheres, said microspheres comprising etoricoxib, wherein the microspheres are coated with a flavored taste-masking coating solution in a one-step coating process, the flavored taste-masking coating solution comprising the following ingredients:
(a) hydroxypropylmethyl cellulose,
(b) polymethacrylate,
(c) mannitol,
(d) aspartame,
(e) artificial cherry flavor,
(f) monoglyceride, and
(g) water.
   prepared by a process comprising:
(1) preparing the flavored taste-masking coating solution as follows:
   (a) dissolving hydroxypropylmethyl cellulose in deionized water;
   (b) adding polymethacrylate and homogenizing; and
   (c) adding mannitol, aspartame, artificial cherry flavor and monoglyceride and homogenizing; and

(2) coating the microspheres with the flavored taste-masking coating solution in a one-step coating process. Within this embodiment, the ingredients in the flavored taste-masking solution are combined to produce a solution having following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |

The term “pharmaceutically acceptable core” means any pharmaceutically acceptable core suitable for coating, such as a crystals, particles, granules and microspheres. Methods for making pharmaceutically acceptable cores are well known in the art. For example, microspheres can be made according to the methods taught in U.S. Pat. No. 5,849,223, granted Dec. 15, 1998.

The term “plurality of pharmaceutically acceptable cores” means more than one pharmaceutically acceptable core as defined above.

Etoricoxib is a selective inhibitor of cyclooxygenase-2 which is useful to treat inflammation and pain in a variety of conditions. Etoricoxib is taught in U.S. Pat. No. 5,861,419, granted on Jan. 19, 1999. Methods for making etoricoxib are taught in U.S. Pat. No. 6,040,319, granted on Mar. 21, 2000.

The term “taste-masking agent” means, for example, polymethacrylate (EUDRAGIT), hydropropylmethylcellulose (HMPC), Hydroxypropylcellulose, (HPC) and vinyl pyrrolidone—vinyl acetate co-polymer (PLASDONE).

The term “sweetening agent” means, for example, sugar and aspartame.

The term “flavoring agent” means for example artificial flavor, such as artificial cherry flavor.

The term “bulking agent” means, for example, mannitol, lactose, starch and calcium phosphate.

The term “glidant” means a lubricant, for example, monoglycerides, talc, silicon dioxide and magnesium stearate.

The coated pharmaceutically acceptable cores of the present invention may be administered in a variety of final dosage forms, such as an oral granule formulation, fast disolving tablets and chewable tablet.

In view of the teachings herein, one skilled in the art can readily make the described “flavored taste-masking coating solution”. This solution may be coated on the pharmaceutically acceptable cores using a variety of applications, such as a fluid bed system. Fluid bed systems for coating pharmaceutically acceptable cores are well known in the art, for example, the Glatt GCPG1 fluid bed (Glatt Air Techniques Inc., Ramsey, N.J.) equipped with a Wurster coating insert and an appropriate air diffusion plate as described in the example below.

For purposes of this specification, the term “about” as used to describe the composition of the flavored taste-masking solution means ±5%, preferably ±2% and more preferably ±1%.

Exemplifying the invention are the following non-limiting examples:

### EXAMPLE 1

### Etoricoxib Oral Granule Formulation

TABLE 1
Composition of Flavored Taste-Masking Coating Formulation

| Excipient Name | % wt/wt |
|---|---|
| Polymethacrylate dispersion 30% | 50 |
| Hydropropylmethyl cellulose (HMPC) | 3 |
| Mannitol | 8.5 |
| Aspartame | 1 |
| Artificial Cherry Flavour | 2.5 |
| Monoglycerides | 5 |
| Water | 30 |
| Total | 100% |

Preparation of the Flavored Taste-Masking Coating-Solution

In a suitable container, the HPMC is dissolved in deionized water under constant stirring. The EUDRAGIT (Polymethacrylate dispersion 30%) dispersion is then added to the HPMC solution and homogenized under constant stirring. Mannitol, aspartame, cherry flavor and monoglycerides are then added successively to the mixture that is continuously stirred until a homogenous dispersion is obtained. The coating suspension contains 35% solids with a 5:1 ratio of polymethacrylate to HPMC.

Taste-Masking Coating Process

In order to evaluate the coating process, 500 g of an API containing core suitable for coating are loaded in a Glatt GCPG1 fluid bed (Glatt Air Techniques Inc., Ramsey, N.J.) equipped with a Wurster coating insert and an appropriate air diffusion plate. The Wurster central partition is set at a 7.5 mm height. The spray lance is fitted with a binary nozzle (Schlick #940) assembled with a #12 liquid insert (1.2 mm) and a 2 mm air cap in position #3, (flush setting). The fluidizing air temperature is set at 30° C. and is introduced in the pre-heated coating unit at an initial velocity of 3 m/s. The air velocity will be increased gradually during the progression of the coating process up to 4.5 m/s. The coating solution is sprayed onto the fluidized bed at an atomization pressure of 2 bar and a spray rate set at 2.5 g/min. At the end, 288 g of coating solution was applied to the bed, corresponding to a 20% wt/wt increase of the initial API containing core. The product is then allowed to dry in a fluidized motion for 3 minutes.

What is claimed is

1. A flavored and taste-masked pharmaceutical composition comprising a plurality of pharmaceutically acceptable cores, said pharmaceutically acceptable cores comprising etoricoxib, wherein the pharmaceutically acceptable cores are coated with a flavored taste-masking cooling solution in a one-step coating process, said flavored taste-masking coating solution comprising the following ingredients:
(a) at least one taste-masking agent and
(b) at least one sweetening agent or at least one flavoring agent or at least one of both.

2. The pharmaceutical composition according to claim 1 wherein the flavored taste-masking coating solution further comprises:
(a) at least one bulking agent, and
(b) at least one glidant.

3. The pharmaceutical composition according to claim 1 wherein the pharmaceutically acceptable cores are microspheres.

4. The pharmaceutical composition according to claim 1 wherein the flavored taste-masking coating solution comprises the following ingredients:
(a) hydroxypropylmethyl cellulose,
(b) polymethacrylate,
(c) mannitol,
(d) aspartame,
(e) artificial cherry flavor,
(f) monoglyceride, and
(g) water.

5. A flavored and taste-masked pharmaceutical composition in accordance with claim 1 comprising a plurality of microspheres, said microspheres comprise b, wherein the microspheres are coated with a flavored taste-masking coating solution in a one-step coating process, the flavored taste-masking coating solution comprising the following ingredients:
(a) hydroxypropylmethyl cellulose,
(b) polymethacrylate,
(c) mannitol,
(d) aspartame,
(e) artificial cherry flavor,
(f) monoglyceride, and
(g) water.

6. The pharmaceutical composition according to claim 5 wherein the ingredients in the flavored taste-masking solution are present in the following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |

7. The pharmaceutical composition according to claim 1 prepared by a process comprising:
(1) preparing the flavored taste-masking coating solution by combining the following ingredients:
(a) at least one taste-masking agent and
(b) at least one sweetening agent or at least one flavoring agent or at least one of both,
(2) coating the pharmaceutical cores with the flavored taste-masking coating solution in a one-step coating process.

8. The pharmaceutical composition according to claim 7 wherein step 1) for preparing the flavored taste-masking coating solution further comprises adding the following ingredients:
(a) at least one bulking agent, and
(b) at least one glidant.

9. The pharmaceutical composition according to claim 7 wherein the pharmaceutical cores are microspheres.

10. The pharmaceutical composition according to claim 7 wherein the flavored taste-masking coating solution is prepared by combining the following ingredients:
(a) hydroxypropylmethyl cellulose,
(b) polymethacrylate,
(c) mannitol,
(d) aspartame,
(e) artificial cherry flavor,
(f) monoglyceride, and
(g) water.

11. The pharmaceutical composition according to claim 10 wherein the flavored taste-masking coating solution is prepared as follows:
(a) dissolving hydropropylmethyl cellulose in deionized water;
(b) adding polymethacrylate and homogenizing; and
(c) adding mannitol, aspartame, artificial cherry flavor and monoglyceride and homogenizing.

12. The pharmaceutical composition according to claim 11 wherein the ingredients in the flavored taste-masking solution are combined to produce a solution having following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |

13. The pharmaceutical composition according to claim 7 wherein the pharmaceutical cores are coated using a fluid bed system.

14. The pharmaceutical composition according to claim 13 wherein the pharmaceutical cores are microspheres.

15. The pharmaceutical composition according to claim 5 prepared by a process comprising:
(1) preparing the flavored taste-masking coating solution as follows:
(a) dissolving hydropropylmethyl cellulose in deionized water;
(b) adding polymethacrylate and homogenizing; and
(c) adding mannitol, aspartame, artificial cherry flavor and monoglyceride and homogenizing; and
(2) coating the microspheres with the flavored taste-masking coating solution in a one-step coating process.

16. The pharmaceutical composition according to claim 15 wherein the ingredients in the flavored taste-masking solution are combined to produce a solution having following amounts:

| Excipient Name | % wt/wt of coating solution |
|---|---|
| Polymethacrylate | about 15 |
| Hydropropylmethyl cellulose (HPMC) | about 3 |
| Mannitol | about 9 |
| Aspartame | about 1 |
| Artificial Cherry Flavour | about 3 |
| Monoglycerides | about 5 |
| Water | about 65 |
